(19) United States
(12) Patent Application Publication
(10) Pub. No.: US 2023/0270930 A1
(43) Pub. Date: Aug. 31, 2023

(54) DRAINAGE DEVICE FOR DRAINING FLUIDS AND/OR WOUND SECRETION FROM THE THROAT AREA, NOSE AREA OR EAR AREA OF A PATIENT, IN PARTICULAR FOR USE AFTER A SURGICAL INTERVENTION IN THE CERVICAL AREA OF THE PATIENT OR IN NEUROLOGY

(71) Applicant: PFM MEDICAL AG, Koeln (DE)

(72) Inventors: Umberto MARIANI, Capriate (IT); Aurel SCHOELLER, Bornheim (DE)

(21) Appl. No.: 18/007,376

(22) PCT Filed: Jul. 7, 2021
(86) PCT No.: PCT/EP2021/068827

§ 371 Date: Jan. 30, 2023

(30) Foreign Application Priority Data
Jul. 31, 2020 (IT) 102020000018787

(51) Int. Cl. A61M 1/00

(52) U.S. Cl. CPC A61M 1/84; A61M 1/64

(57) ABSTRACT

A method for operating an elastically mounted forming machine which is path-bound or force-dependent, in which method a working stroke of a ram device operatively connected to the drive is initiated by means of a drive, and a predefined forming process is carried out on a workpiece by moving the ram device during said working stroke, in particular due to the interaction of an upper tool located on the ram device with a lower tool located on a tool table, wherein the inertial forces and/or moments of inertia occurring during operation owing to the initiation of the working stroke and/or owing to an imbalance in the drive are at least partially compensated. The method, wherein at least one kinematic variable (s(t),v(t),a(t)) of a rigid body motion of the elastically mounted forming machine is detected during the operation thereof, wherein the time at which the working stroke is initiated is adapted to an instantaneous phase position of the at least one kinematic variable (s(t),v(t),a(t)) of the rigid body motion in order to generate inertial forces and/or moments of inertia so as to counteract the rigid body motion of the forming machine.

### FIELD

The invention relates to a method for operating an elastically mounted forming machine which is path-bound or force-controlled, method in which a working stroke of a ram device operatively connected to the drive is carried out by means of a drive, and a predefined forming process is carried out on a workpiece by a motion of the ram device during the respective working stroke, in particular in interaction of an upper tool located on the ram device with a lower tool located on a tool table, wherein the inertial forces and/or moments of inertia occurring during operation due to the initiation of the working stroke and/or due to an imbalance in the drive are at least partially compensated.

### BACKGROUND

The operation or the control of such an elastically mounted path-bound or force-controlled forming machine is well-known in this field. Such machines can be configured, depending on the embodiment, for pressure forming, tensile compressive forming, bending or thrust forming. For path-bound forming machines, for example path-bound presses, the travel of the ram device (bear) is determined by the kinematics of the drive of the machine. The drive is generally carried out by an electric motor that drives a flywheel that can be connected to the ram device by clutch engaging for initiating the working stroke. Force-controlled forming machines have a controllable drive, in particular a hydrostatic drive, for example as a pump, or a servomotor drive by which a ram device permanently operatively connected therewith is moved after the respective activation of the drive for initiating the working stroke for carrying out the forming process. The time of initiation and the carrying out of the working stroke of operator-controlled machines generally takes place by means of a so-called two-hand activation.

It is common to path-bound as well as to force-controlled forming machines that they are exposed to occurring inertial forces and/or moments of inertia during the carrying out of the forming process. They can be caused, for example, with permanently rotating drive shafts, by clutch engaging and disengaging of the ram device or by an imbalance in the drive of a force-controlled forming machine and result in a tumbling and/or tilting motion of the forming machine. Such motions can cause an increased mechanical stress of the machine and possibly result in a non-compliance of the dimensional tolerances at the formed workpiece. The generated inertial forces or moments of inertia cause, because of an elastically bearing of the forming machine on a supporting foundation, for example on a foundation soil, a excitation of the rigid body modes of this vibratory system that are defined by the forming machine and the elastic bearing, which causes the described tumbling and/or tilting motion of the machine relative to the foundation on which the forming machine bears with an elastic bearing.

Active measures for acting on the undesired whole body movements of such a forming machine are known in this field, in particular the eradication of vibration phenomena by installing an appropriately adapted additional vibratory system, designated in this field as a damper, on the mass to be steadied. For example, the published patent application DE 10 2008 046 763 A1 relates to a generic high-speed press with inertial moment compensation for which a counterweight is provided that serves for taking over the reactive power of the ram, wherein the phase of motion of the counterweight is adapted to the phase of motion of the ram. The published patent application DE 2806584 relates to an also generic eccentric press on which a compensating device with a movable mass part is placed for mass balancing, that has driving elements for driving the mass part in antiphase with preserved angles relative to the eccentric shaft.

The providing of such compensating devices for the respective forming machine is connected to an increased expenditure on the device.

JP 2008-290126A relates to the forming operation with a press. This press is supported by a spring damper system on a foundation. The travel of the main frame is detected by a displacement sensor. Moreover, a speed sensor is provided for detecting the first time derivation. The angular speed and the angular position of the crank drive are the control variables that are influenced to reduce the vibrations on the press.

The aim of this invention is, for a conventional elastically mounted forming machine, to make available at least an attenuation of the described rigid body motion of the forming machine in operation without a significantly expenditure on equipment having to be provided as in the prior art.

### SUMMARY

With the method according to the invention, a working stroke of a ram device operatively connected to the drive is carried out, working stroke with which a predefined forming process is carried out on a workpiece due to a motion of the ram device during the respective working stroke, in particular in interaction of an upper tool located on the ram device with a lower tool located on a tool table, wherein the inertial forces and/or moments of inertia occurring during operation due to the initiation of the working stroke and/or due to an imbalance in the drive are at least partially compensated. The method according to the invention is characterized in that at least one kinematic variable of a rigid body motion of the elastically mounted forming machine is detected during the operation thereof, in particular continuously detected, wherein the time at which the working stroke is initiated is adapted to an instantaneous phase position of the at least one kinematic variable, in particular path s(t), speed v(t) and/or acceleration a(t) of the rigid body motion for generating inertial forces and/or moments of inertia so that the rigid body motion of the forming machine is counteracted.

The method according to the invention results in a reduction of the amplitude of a tumbling and/or tilting motion of the forming machine. Such a tumbling or tilting motion of the forming machine substantially constitutes a rigid body motion of the forming machine excited by the occurring inertial forces and/or moments of inertia in a vibratory system formed by the forming machine itself as well as an elastic bearing device with which the forming machine is elastically mounted on a supporting foundation. According to the invention, this vibration reduction can be made available without a significantly increased expenditure on the device being necessary as in the prior art.

Instead, in the method according to the invention, the control of the initiation of the working stroke takes place depending on a present rigid body motion of the forming machine in the vibratory system so that the inertial forces and/or moments of inertia are introduced into the vibratory system of the rigid body motion of the forming machine by a phase-exact initiation of the working stroke caused by the clutch engaging of the ram device and/or by an imbalance in the drive in such a manner that they counteract the present rigid body motion of the forming machine. The excitation of the rigid body motion can take place due to the occurring inertial forces and/or moments of inertia, and be for example impact-type with a predefined amplitude and duration.

In case of operator-controlled machines, it can insofar be provided that, after a two-hand activation by the operator, the above mentioned method according to the invention is carried out for controlling the operation of the forming machine, i.e. after the two-activation, at least one kinematic variable of the rigid body motion of the elastically mounted forming machine is detected during the operation thereof and the time of the initiation of the working stroke is adapted to an instantaneous phase position of the at least one kinematic variable of the rigid body motion in order to generate inertial forces and/or moments of inertia so that the rigid body motion of the forming machine is counteracted. According to the invention, it can however also be provided for operator-controlled machines that the method according to the invention for controlling the operation of the forming machine is permanently carried out or permanently runs after a start of operation of the forming machine during which at least one kinematic variable of the rigid body motion of the elastically mounted forming machine is detected and the time of the initiation of the working stroke is adapted to an instantaneous phase position of the at least one kinematic variable of the rigid body motion in order to generate inertial forces and/or moments of inertia so that the rigid body motion of the forming machine is counteracted and wherein the working stroke is only initiated after a two-hand activation has been carried out by the operator for the working stroke to be initiated. This being, a control device of the forming machine that is equipped and configured for the control of the operation according to the invention of the forming machine can detect a signaling induced by the two-hand activation of the operator, in particular as an electric signal, and process it for implementing the method according to the invention.

The method according to the invention thus results in a significantly reduced deflection of the forming machine compared to an uncontrolled mode of operation. The method according to the invention for operating the elastically mounted forming machine makes possible the faster achievement of a state of the forming machine with a lower motion elongation or amplitude. Thus, a subsequent working stroke can possibly start earlier with the advantage of a faster cycle sequence during operation which can be advantageous in particular for an automatic feed of the forming machine for which the respective working piece has to be positioned accurately. Moreover, because of the lower stress of the elastic bearings of the forming machine, the lifetime of present bearings can be increased or allows for the use of bearings with smaller dimensions.

It should be noted that the at least one kinematic variable of a rigid body motion of the elastically mounted forming machine can be, for example, a path or angle deflection from a respective rest position, a time derivation of these variables or the corresponding results of a numerical simulation of the rigid body motion of the forming machine, carried out previously or simultaneously with the operation of the forming machine, for determining a respective speed and/or acceleration. Appropriate kinematic variables can be defined in particular with respect to the different modes of the system for the description of the motion of the elastically mounted forming machine. The method according to the invention can basically be applied to all the degrees of freedom of the rigid body motion of the forming machine.

Accordingly, it is within the scope of the invention that the detection of the at least one kinematic variable of the rigid body motion is carried out for example by means of a measurement with a motion sensor and/or by means of a calculation, in particular within the scope of a simulation of the rigid body motion of the forming machine.

Further advantageous characteristics and further developments of the invention are indicated in the following general description, in the figures, in the description of the figures as well as in the subclaims.

It can appropriately be provided that the operation of the forming machine is controlled by a machine control that activates the drive for carrying out the forming process, possibly after the presence of a two-hand activation signal induced by an operator at the time of the initiation of the working stroke and/or activates a coupling device located between the drive and the ram device for establishing an operative connection between the drive and the ram device for carrying out the forming process. In the first case, the matter can be for the forming machine of a force-controlled forming machine for which the drive is permanently connected to the ram device and in the second case of a path-bound forming machine for which an operative connection can be established between the drive and the ram device by clutch engaging a controllable coupling and can be released upon clutch disengaging.

As explained, the elastically mounted forming machine can carry out a rigid body motion excited by the occurring inertial forces and/or moments of inertia. It has become apparent, in particular during the operation of stroke-forced forming machines, for which the excitation, in particular an excitation of the rigid body motion, is carried out substantially during the initiation of the working stroke or during clutch engaging, that a time range can be appropriately selected for adjusting an optimal time for initiating the working stroke or for clutch engaging the ram device, time range within which a global maximum of the first time derivation of the course of a deflection of the forming machine is situated.

The clutch engaging time is preferably situated immediately before reaching this global maximum. With respect to a harmonious vibration, immediately before can mean, depending on the embodiment, <30°, <20°, in particular <10°, before reaching this global maximum in the first time derivation of the course of the deflection of the forming machine. It can also be provided that the excitation moment exactly coincides with the reaching of this global maximum in the first time derivation of the course of the deflection of the forming machine. Basically, the time of clutch engaging of the ram device or of the initiation of the working stroke is selected so that a motion of the forming machine is induced to a direction that is in opposite direction of the temporarily deflection of the forming machine, i.e. the excitation should take place in phase opposition.

It became apparent in particular during the operation of force-controlled forming machines, for which the excitation of the rigid body motion substantially takes place due to an imbalance of the drive during the working stroke and/or the return stroke of the ram device, that a time for initiating the working stroke can appropriately be selected in such a manner that the excitation of the rigid body motion of the forming machine that can take place impulsively in particular during the working stroke and/or the return stroke, takes place due to the occurring inertial forces and/or moments of inertia within a time period in which a global maximum of the first time derivation of the course of a deflection of the forming machine is situated. The time of initiation of the working stroke can preferably take place so that the time of excitation of the rigid body motion is situated immediately before reaching this global maximum. With respect to a harmonious vibration of the rigid body motion that can be assumed approximately in cases of a rigid body motion, “immediately before” can mean, depending on the embodiment, <30°, <20°, in particular <10°, before reaching this global maximum in the first time derivation of the course of the deflection of the forming machine. It can also be provided that the time of excitation exactly coincides with the reaching of this global maximum in the first time derivation of the course of the deflection of the forming machine. Basically, the time of the excitation of the rigid body motion is selected so that a motion of the forming machine is generated into a direction that is in opposite direction of the temporarily deflection of the forming machine, i.e, the excitation should take place in phase opposition.

In order to make available the information necessary for the control of the time of clutch engaging the ram device or of the initiation of the working stroke, the at least one kinematic variable of the rigid body motion of the forming machine relative to the supporting foundation is detected by at least one motion sensor. This motion sensor can be configured, for example, as a deflection sensor such as a displacement sensor, a speed sensor or an acceleration sensor. Depending on the embodiment, the motion sensor can be placed in particular on the forming machine itself or on the bearing device. The motion sensor, in particular the acceleration sensor, can detect the magnitude of an elastic deformation on the bearing device, for example of an elastic deformation of an elastomer from which the at least one kinematic variable of the rigid body motion of the forming machine can be determined, in particular can be calculated, for the optimal temporal clutch engaging of the ram device or the initiation of the working stroke.

A multitude of sensors are basically possible as motion sensors for implementing the method according to the invention that detect in particular a deflection, a speed and/or an acceleration of the rigid body motion of the forming machine. The motion sensor can be configured, for example, as a plunger coil sensor or as a resistance sensor. It is also possible to configure the motion sensor as an optical sensor.

A respective output signal of the at least motion sensor is supplied as an input signal of a machine control of the forming machine for making available a corresponding signal to a control device associated to the forming machine, wherein this signal supply can also be wireless. Preferably, several motion sensors can be provided that detect, in particular that measure, one or several kinematic variables of the rigid body motion of the forming machine and, for example make them available to a central control device such as a machine control of the forming machine for carrying out the method according to the invention.

In particular in such embodiments in which the forming machine carries out a complex rigid body motion, it can appropriately be provided that the at least one kinematic variable of the rigid body motion of the forming machine relative to the supporting foundation is calculated on the base of a rigid body simulation model and the time of clutch engaging or of the initiation of the working stroke is defined depending on a measured and of the calculated instantaneous value of the at least one kinematic variable. For example, the time of clutch engaging or of the initiation of the working stroke can be adjusted basically after the calculated kinematic variable, wherein the measured instantaneous value is used as a control variable, wherein, in presence of a predefined difference value, the control is interrupted for safety reasons and the operation completed. It is however possible that the time of clutch engaging or of the initiation of the working stroke is adjusted after the measured kinematic variable, wherein the calculated instantaneous value is used as a control variable, wherein, in presence of a predefined difference value, the control is interrupted for safety reasons and the operation completed.

It can be provided that the at least one kinematic variable of the rigid body motion of the forming machine is calculated relative to the supporting foundation on the base of a rigid body simulation model of the elastically mounted forming machine and the time of the initiation of the working stroke or of the clutch engaging of the ram device is defined depending on an instantaneous value of the at least one kinematic value calculated through the simulation. For coupling the simulation with the real operation of the forming machine, it can preferably be provided that a kinematic variable of the rigid body motion of the elastically mounted forming machine relative to the supporting foundation is measured and a synchronization signal is derived depending on the measurement signal and/or on an operation signal from a machine monitoring device, synchronization signal with which the time sequence of the kinematic variable calculated with the simulation model is synchronized with the real rigid body motion of the forming machine.

It can also be provided to use output signals of a machine monitoring device provided for conventional forming machines that can be arranged in particular for measuring one or several kinematic variables of the rigid body motion of the forming machine or of other operating parameters, together with results of a simulation of the rigid body motion of the forming machine, in order to define the time of clutch engaging the ram device or of the initiation of the working stroke as described in such a manner that the inertial forces and/or moments of inertia introduced into the vibratory system counteract the instantaneous rigid body motion of the forming machine. Thus, the method according to the invention can be carried out for controlling an elastically mounted forming machine without additional expenditure on equipment compared to a conventional method for controlling a conventional forming machine.

Depending on the specific forming machine and the operation thereof, the forming machine can also be exposed to elastic deformations relative to certain sections or components of the machine while executing a working stroke. In order to at least partially reduce such elastic deformation movements for the control according to the invention of the operation of the forming machine, it can be provided to detect a variable of an elastic deformation of a predefined section or component of the forming machine such as a deflection relative to the housing or to a machine foundation of the forming machine, wherein the time of clutch engaging the ram device or of the initiation of the working stroke is adapted to an instantaneous phase position of the one variable of the deformation movement of the predefined section or component of the forming machine for generating inertial forces and/or moments of inertia during clutch engaging or initiating the working stroke so that the elastic deformation movement of the predefined section or of the component of the forming machine is counteracted. Such a section or such a component can comprise, for example, a damping element such as an elastomer body or a shock absorber or even an elastic bend line section resulting from the occurring stress within the forming machine that is thus located in the forming machine and insofar differs from the bearing device by which the forming machine is elastically supported on the supporting foundation.

It can be provided to reduce the rigid body motion of the forming machine as described by the phase-exact clutch engaging of the ram device or the initiation of the working stroke as well as similarly an elastic deformation movement within the forming machine, wherein the movement reduction is set by priority to the rigid body motion of the forming machine or to the elastic deformation movement of a section or of a component of the forming machine, or a compromise is reached to reduce both movements or deflections in roughly equal measure. This being, in equal measure can mean that the respective deflection amplitudes of the rigid body motion of the forming machine and the elastic deformation are substantially equally reduced within the machine.

The method according to the invention for operating or for controlling the operation of a forming machine results in a reduction of a rigid body motion of the forming machine or of an elastic deformation of a section or of a component of the forming machine and can be used in particular to adjust a higher working cycle rate compared to a conventional method so that a higher production rate of the formed products can be achieved when implementing the method according to the invention. It can be provided that in operation a present amplitude value, that has been measured by means of a motion detector or a sensor or that has been calculated through a simulation, of the at least one kinematic variable of the rigid body motion of the forming machine, for example of a deflection into a predefined direction, is compared to a predefined amplitude threshold, and the cycle rate of the forming machine is increased when the present amplitude value is lower than the predefined threshold. The method according to the invention for operating a forming machine can also comprise a regulation of such a deflection amplitude for which the described threshold of the deflection amplitude is used for example as a reference variable and the cycle rate for the working stroke can represent a control variable of the regulation.

Furthermore, the invention relates to a forming machine, in particular to a press. For example, such a forming facility can be a path-bound forming facility that can have, depending on the embodiment, a crank gear or a cam gear. In particular in the design of a press, the drive can comprise an electric motor that drives a flywheel that delivers the energy by means of a coupling device to the main gear, as described a crank gear or a cam gear. Exemplary forming devices are slider crank presses, eccentric presses and toggle presses. Moreover, the forming device according to the invention can also be a force-controlled forming machine, wherein the drive can be made available as a direct drive, for example either by an electric servomotor, also called torque motor, or comprises a hydrostatic drive for which the energy stored in the pressure medium is converted into mechanical energy by means of cylinders over a pump drive. In the nomenclature of this invention, a ram device that carries an upper tool of the machine is moved by the drive in case of force-controlled forming machines as well as of the path-bound forming machines.

### BRIEF DESCRIPTION OF THE DRAWINGS

The invention shall be explained below with the description of an embodiment and of modifications thereof with reference to the accompanying drawings.

FIG. 1 shows a front view of a forming machine according to the invention as a press for carrying out the method according to the invention.

FIG. 2 shows a symbolic representation of a tilting/tumbling motion of the machine of FIG. 1.

FIG. 3 shows a process chart of an embodiment of the method according to the invention.

FIG. 4a shows the time sequence of a deflection s(t) and the time derivation thereof v(t) of the forming machine during the rigid body motion thereof in operation when carrying out the method according to the invention for a phase-optimized clutch engaging of the ram device according to the invention.

FIG. 4b shows a representation corresponding to FIG. 4a for a non phase-optimized clutch engaging of the ram device.

### DETAILED DESCRIPTION

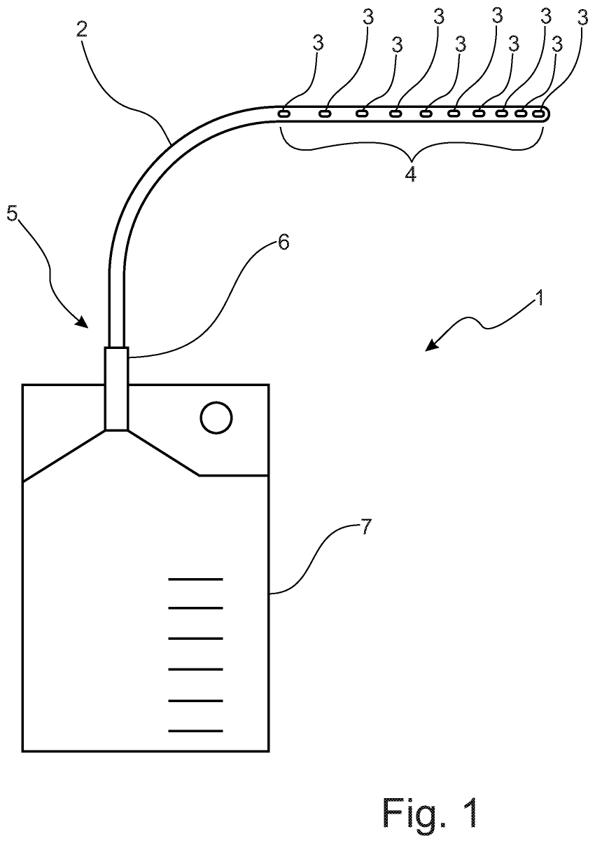

FIG. 1 shows in a front view the structure of an elastically mounted press 1 that is designed and configured according to the invention so as to carry out the method according to the invention for controlling the operation of an elastically mounted forming machine. This forming machine comprises a stand 2 that bears on a machine frame 3. A drive that comprises here an electric motor 4a and an oscillating mass 4b driven by the motor cooperates in the described embodiment with a ram device 5 or bear over a controllable coupling device, hidden in the figure, wherein an operative connection can be adjusted between the drive 4a, 4b and the bear 5 over the controllable coupling for carrying out the working stroke and can be disengaged for preparing the next working stroke. The ram device carries at its end an upper tool 6 that cooperates with a lower tool 7 that is located on the machine frame 3, for implementing a forming process of a workpiece that is not represented. The machine frame carries the drive and the bear via the stands 2 and bears itself on the foundation soil or on the supporting foundation 9 by means of several elastic bearing elements 8, each comprising in the described embodiment an elastomer body. In another embodiment, in particular in a path-bound forming machine, it can also be provided that the press comprises a drive with a servomotor that is rigidly connected to the ram device 5.

In all the embodiments of such forming machines, a tumbling and/or tilting motion of the press 1 is generally generated during the clutch engaging for connecting the drive and the ram device or during the initiation of the working stroke and/or during the carrying out of the working stroke, for example because of an imbalance in the drive, due to the respective occurring of inertial forces or moments in inertia.

For force-controlled forming machines, these inertial forces or moments of inertia that excite a rigid body motion of the forming machine can be generated in particular by an imbalance in the drive and insofar occur during the entire time range of a working stroke of the ram device of the forming machine. For path-bound forming machines, these inertial forces or moments of inertia that excite a rigid body motion of the forming machine can occur in particular during the clutch engaging of the coupling located between the drive and the ram device or during the initiation of the working stroke. In such cases in which the drive experiences an imbalance, additional excitation torques or excitation forces can occur.

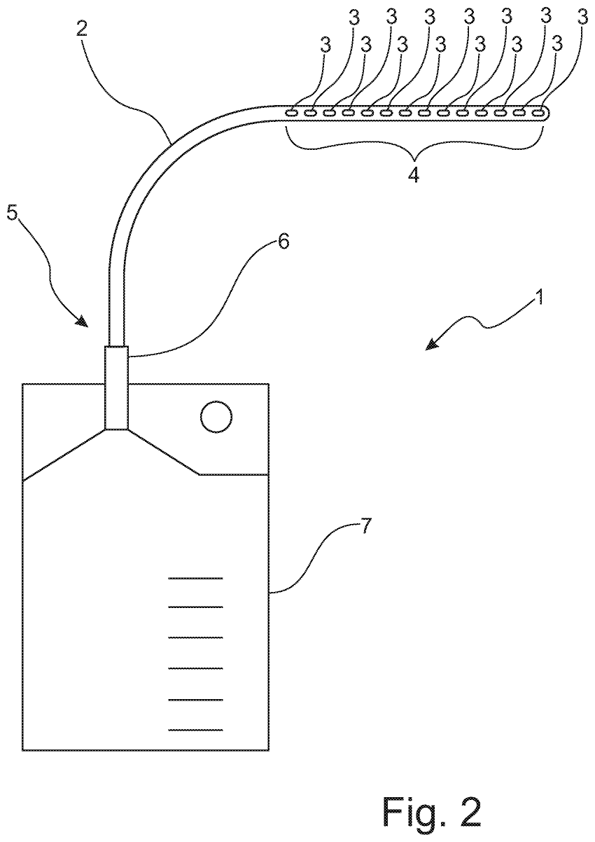

FIG. 2 shows a symbolic representation of a possible tilting motion K of the press of FIG. 1 as a vibration. Possible modes of a rigid body motion of a vibratory system that is formed by the press 1 supported on the foundation soil 9 by the elastic bearing elements 8 can basically be excited.

As explained, the press of FIG. 1 is configured as a path-bound forming machine for which the inertial forces or moments of inertia that excite a rigid body motion of the forming machine are caused during the clutch engaging of the coupling for adjusting an operative connection between the drive and the ram device. The operation of the forming machine of FIG. 1 is controlled in the described embodiment by a machine control that controls, at the time of the initiation of the working stroke, the coupling device located between the drive and the ram device for establishing an operative connection between the drive and the coupling.

It is essential for carrying out the method according to the invention or the operation of the forming machine according to the invention that at least one kinematic variable of the rigid body motion, for example a deflection, of the elastically mounted forming machine 1 is detected during the operation thereof, is here measured by corresponding sensors as one or several motion sensors, wherein the time of the initiation of the working stroke, here the time for causing the operative connection between the drive and the ram device is adjusted in such a manner that the inertial forces and/or moments of inertia generated during the clutch engaging counteract the rigid body motion of the forming machine. In another embodiment, it can also be provided that the at least one kinematic variable of the rigid body motion, for example a deflection, is calculated by simulation of the rigid body motion of the forming machine, wherein an output signal of a motion sensor can be used for detecting the motion of the forming machine or another operating signal for the synchronization of the real motion of the forming machine with the simulation.

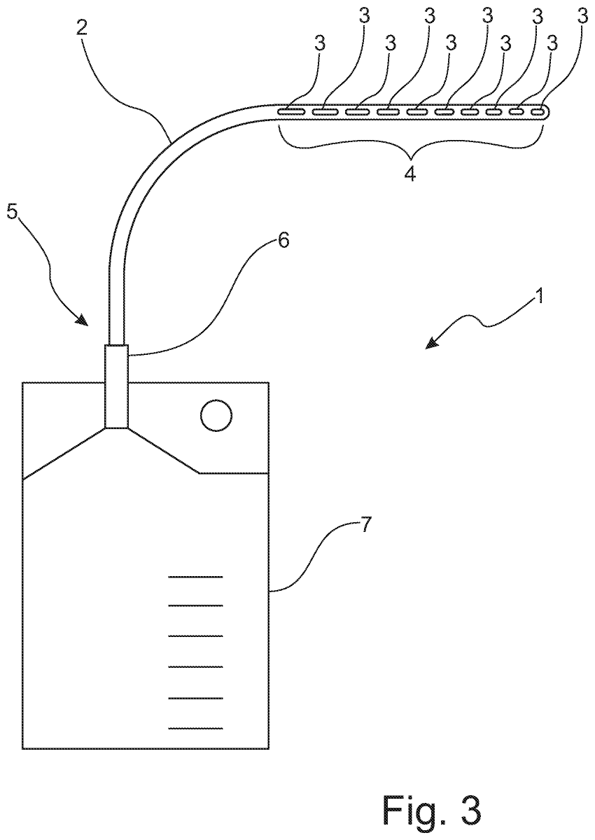

The method according to the invention for the phase-exact clutch engaging of the coupling of the press indicated in FIG. 1 is indicated in FIG. 3 and is carried out in the described embodiment by a central machine control of the forming machine. This being, it is started from the fact that the forming machine is in operating mode for which, after a release signal is available, in particular a two-hand activation signal induced by an operator, a working stroke of the ram device is initiated with the clutch engaging, working stroke during which a predetermined forming process is carried out on a workpiece in interaction of the ram device or of the upper tool carried thereby with a lower tool located on a tool table, wherein the ram device is returned in a subsequent return stroke, and the operative connection between the drive and the ram device is suppressed by clutch disengaging until a further working stroke is initiated by clutch engaging after a further release signal is available.

In the described embodiment, the clutch engaging is adapted to a phase position of a kinematic variable, here a deflection of the forming machine from the rest position. The starting point of the method steps indicated in FIG. 3 is an operating situation for which the drive is decoupled from the ram device and after a release signal is made available, the time of the clutch engaging is to be fixed, wherein the forming machine carries out a rigid body motion caused by preceding excitations that is carried out, depending on the embodiment, differently attenuated in particular because of damping properties of the bearing elements. It should be noted that when carrying out the method according to the invention with a fully automated forming machine, the checking for the presence of a release signal may be dispensed with.

In the method steps of FIG. 3, in step 100 the present deflection of the rigid body motion of the forming machine 1 is measured by a motion sensor, wherein the machine control is designed to check in step 110 if the first time derivation of the course of the deflection, i.e. if the speed is situated in the range of a global maximum of the rigid body motion. As far as this is not the case, there does not take place any clutch engaging of the ram device for adjusting an operative connection between the drive and the ram device; instead, a jump back to step 100 takes place, i.e. for carrying out a further measurement of the deflection of the forming machine. This measurement and check loop will run until the speed is situated in the range of the global maximum of the speed determined in the course indicated in FIG. 3 prior to the start so that an initiation of the working stroke substantially in phase opposition can take then place in step 120 with a phase-adapted introduction of the excited inertial forces and/or moments of inertia so that the present rigid body motion of the forming machine is counteracted. With the initiation of the working stroke in step 120, the carrying out thereof takes place in step 130 for carrying out a predetermined forming process; thereafter, the return stroke of the ram device and the disengagement of the operative connection between the ram device and the drive takes place in step 140 for preparing a further working stroke. As far as the end of operation is reached, the forming machine is stopped; otherwise a jump into the start of the measuring loop, i.e. to step 100, takes place.

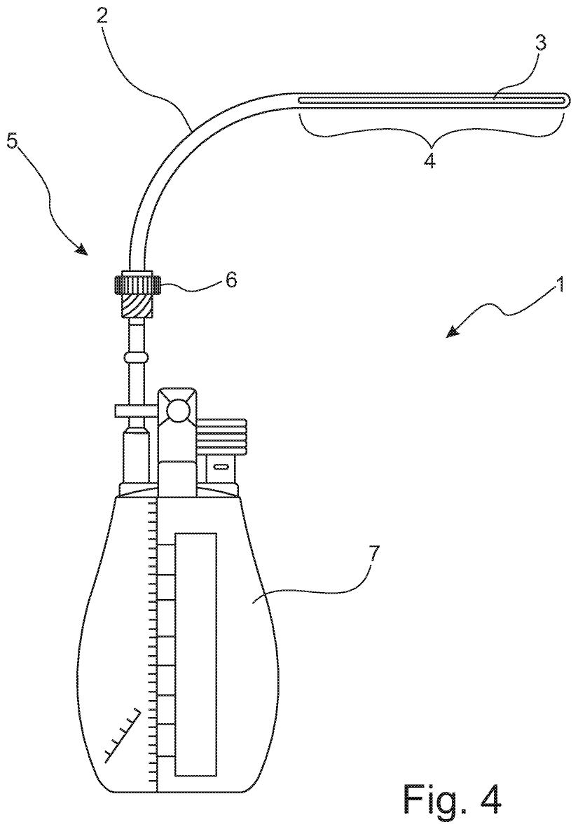

Exemplary courses of the rigid body motion of the forming machine of FIG. 1 are indicated in the FIG. 4a, 4b. This being, the respective upper graph shows the course of a deflection of the forming machine in operation and the lower time course shows the resulting speed of the deflection. In both figures, the time courses show prior to the time TO or TO′ the rigid body motion of the forming machine 1, the operative connection between the drive and the ram device being disengaged, this resulting in a weakly damped vibration. At the time TO or TO′, a design-related and application-related excitation, here an approximately impact-type excitation, takes place over a time period (T1−T0) or (T1′−T0′) due to the establishing of the operative connection between the drive and the ram device via the clutch engaging of the coupling so that the vibratory system, that is formed by the forming machine 1 elastically mounted by means of elastic bearing elements 8 on the supporting foundation 9, receives excitation energy.

The representations of FIG. 4a, 4b show the time courses during and after the introduction of inertial forces or moments of inertia during the clutch coupling of the ram device. The external excitation of the rigid body motion recognizably takes place for the courses of FIG. 4a at a time at which the speed of the rigid body motion is approximately maximal. Furthermore, the excitation occurs for generating an opposite-phase motion of the forming machine and results in a subsequent motion of the forming machine with a reduced amplitude. By contrast, FIG. 4b shows the result of an excitation that is identical with that of FIG. 4a, wherein however the time of the clutch engaging is indeed again situated after a global maximum of speed has been reached, but the excitation is carried out in phase with the present deflection so that, after the disturbance has subsided, there results a rigid body motion with a considerably higher amplitude compared to the situation of FIG. 4a.

The curves of FIGS. 4a and 4b show the efficiency of the method according to the invention and/or of the forming machine configured according to the invention for reducing a rigid body motion of the elastically mounted forming machine in operation. Depending on the embodiment, several excitations can also occur within an operating cycle of the forming machine at different times; in these cases the method according to the invention can basically also be applied for reducing a rigid body motion of the forming machine with the above described advantages.

### LIST OF REFERENCE NUMERALS

1 Forming machine, press
2 Stand
3 Machine foundation, machine frame, machine housing
4a Electric motor
4b Oscillating mass
5 Ram device, bear
6 Upper tool
7 Lower tool
8 Elastic bearing element, bearing device
9 Foundation soil, supporting foundation
K Tilting motion
s Deflection, stroke
v Speed
T0, T0′ Time of initiation of a working stroke or adjustment of an operative connection between the drive and the ram device
T1, T1′ Disengagement of the operative connection

What is claimed is:

1-10. (canceled)

11. A drainage device configured to drain fluid and/or wound secretion from a cervical or neurological region of a patient, the drainage device comprising:
a flexible drainage tube with one or more drainage openings, insertable into the cervical or neurological region of the patient, and
wherein the one or more drainage openings are arranged over a length of 15 cm to 25 cm at a distal end of the drainage tube.

12. The drainage device according to claim 11, wherein the drainage device is configured to drain the fluid and/or wound secretion after a surgical procedure in an ear, nose or throat region of the patient.

13. The drainage device according to claim 11, wherein the one or more drainage openings are arranged over a length of 19 cm to 21 cm at the distal end of the drainage tube.

14. The drainage device according to claim 11, wherein the drainage tube has an outer diameter of 3 mm to 8 mm.

15. The drainage device according to claim 11, wherein the drainage tube has an inner diameter of 1 mm to 6 mm.

16. The drainage device according to claim 11, wherein the drainage tube has a length of 30 cm to 100 cm.

17. The drainage device according to claim 11, wherein the drainage tube has multiple drainage openings spaced from each other.

18. The drainage device according to claim 17, wherein a spacing between the respective drainage openings along the length of the drainage tube is constant.

19. The drainage device according to claim 17, wherein a spacing between the respective drainage openings along the length of the drainage tube is irregular.

20. The drainage device according to claim 17, wherein a size of the drainage openings increases in a direction of the distal end of the drainage tube.

21. The drainage device according to claim 17, wherein a spacing between the drainage openings decreases in a direction of the distal end of the drainage tube.

22. The drainage device according to claim 17, wherein the multiple drainage openings each have a length of 2 mm to 6 mm.

23. The drainage device according to claim 17, wherein the multiple drainage openings each have a width of 1 mm to 4 mm.

24. The drainage device according to claim 17, wherein the multiple drainage openings are arranged at opposite sides of the drainage tube.

25. The drainage device according to claim 11, wherein the proximal end of the drainage tube has a connector to connect the drainage tube to a container to collect the fluid and/or wound secretion.

26. The drainage device according to claim 11, further comprising a container to collect the fluid and/or wound secretion.

27. The drainage device according to claim 26, wherein the container is a bag.

28. The drainage device according to claim 26, wherein the container is a vacuum container.

29. The drainage device according to claim 11, wherein the distal end of the drainage tube is at least one of atraumatic and rounded.

30. A method of treating a patient, comprising:
inserting a flexible drainage tube with one or more drainage openings into a cervical or neurological region of the patient, wherein the flexible drainage tube is of a drainage device configured to drain fluid and/or wound secretion from the cervical or neurological region of a patient; and
wherein the one or more drainage openings are arranged over a length of 15 cm to 25 cm at a distal end of the drainage tube.
